(19) United States
(12) Patent Application Publication
(10) Pub. No.: US 2021/0290098 A1
(43) Pub. Date: Sep. 23, 2021

(54) WEARABLE BODY COMPOSITION ANALYZER

(71) Applicant: Rowan University, Glassboro, NJ (US)

(72) Inventors: Adarsh K. GUPTA, Voorhees, NJ (US); Muhammad USMAN, Glassboro, NJ (US); Wei XUE, Haddon Township, NJ (US)

(21) Appl. No.: 17/261,749

(22) PCT Filed: Oct. 28, 2019
(86) PCT No.: PCT/US19/58334

§ 371 Date: Jan. 20, 2021

Related U.S. Application Data
(60) Provisional application No. 62/752,151, filed on Oct. 29, 2018.

(51) Int. Cl. A61B 5/0537; A61B 5/00; A61B 5/263

(52) U.S. Cl. CPC A61B 5/0537; A61B 2562/166; A61B 5/263; A61B 5/0022

(57) ABSTRACT

A device for analyzing body composition provides improved accuracy in measuring body fat, lean body mass and body water using bio-electrical impedance (BIA) measurement. The includes a ring sized to fit a human finger, and including components for passing current. Electrodes are provided for contacting the ring finger of one hand (on an interior portion of the ring), and for making contact with fingers of the opposing hand (on an exterior portion of the ring), so that BIA of the human body can be measured across the human body between the left and right hands, as the result current and voltage applied by the ring. The ring may include components for performing the BIA analysis, or for communicating data to another device for performing the BIA analysis. Results of the analysis may be displayed at the other device, or via a display supported on the ring.

### CROSS-REFERENCE TO RELATED APPLICATION

This application claims the benefit of priority, under 35 USC 119(e) to U.S. Provisional Patent Application No. 62/752,151, filed Oct. 29, 2018, the entire disclosure of which is hereby incorporated herein by reference.

### FIELD OF THE INVENTION

The present invention relates generally to an apparatus and method for analyzing body composition using a wearable device, and more particularly to a finger-wearable device for improving the accuracy of measuring body composition using bio-electrical impedance measurement.

### BACKGROUND

It is well known that the human body is composed primarily of water, protein, bone, and fat, and to a lesser extent, other components. The combined weight of these components makes up the body's weight. For a variety of reasons, it is desirable to measure, and monitor over time, body composition, particularly with respect to the fat component. Quantitatively measuring the respective elements is called body composition analysis.

Various body composition analyzers have been developed for performing body composition analysis. Such analyzers are known to use various known techniques. An exemplary approach for measuring body composition is bioelectrical impedance analysis (BIA). As well-known in the art, this approach generally involves passing a weak alternating current across the human body to analyze the body composition by measuring the electrical resistance or the impedance of the body. See, e.g., K. Aroom, M. S. et al., Bioimpedance Analysis: A Guide to Simple Design and Implementation, 153 Journal of Surgical Research, 23-30 (2009), and U. Kyle et al., Bioelectrical Impedance Analysis—Parts I and II, 23 Clinical Nutrition 1226-1243 and 1430-1453 (2004), the entire disclosures of which each of which are hereby incorporated herein by reference. This is achieved by passing current between electrodes of the analyzer device. One or more sensors of the analyzer device measure the electrical resistance, and various techniques are known in the art for interpreting readings captured by the sensor(s).

The World Health Organization has declared obesity as one of the fastest growing global epidemics. People suffering from obesity are at an increased risk of serious health conditions including stroke, diabetes, high blood pressure, and other chronic diseases caused by excess fat in the body. To study and control this obesity epidemic, precise measurement of the body fat mass is necessary.

It has been recognized that the accuracy and reproducibility of measurements taken using BIA can be undesirably low, particularly when performed by laypersons that are unskilled in taking reliable measurements. It is believed that user error and/or deficiencies in existing analyzer devices contribute to the undesirably low accuracy.

Additionally, most body impedance analyzers currently available on the market are cumbersome and expensive. For example, the full-sized body fat analyzers used in clinics and hospitals can weigh more than 35 kg and occupy over 1 m³ of space. Such analyzers are also very expensive and can easily cost more than $12,000. Furthermore, they are not designed for people with disabilities, especially for those who wear prosthetic legs. Even the off-the-shelf handheld devices (e.g., OMRON body fat monitor HBF 306C) are still relatively large with limited portability. Even though continuous measurement may not be considered absolutely necessary for everyday users, recent research shows that frequent recording of body impedance has a significant impact on patients with effective results for lymphedema treatment (Ridner et al. 2014), hydration content in critically ill patients (Jones et al. 2015), and daily obesity management (Choi et al. 2015a).

What is needed is an apparatus and method for analyzing body composition that provides improved accuracy in measuring body composition using bio-electrical impedance measurement.

### SUMMARY OF THE INVENTION

The present invention provides an apparatus and method for analyzing body composition providing improved accuracy in measuring body fat, lean body mass and body water using bio-electrical impedance measurement. More particularly, the present invention provides a finger-wearable device for improving the accuracy of measuring body composition using bio-electrical impedance measurement.

In an exemplary embodiment, the finger-wearable device comprises a sensing system including a closed-loop or substantially closed-loop ring sized to fit a human finger, and including components for passing current. In some embodiments, the ring comprises components to perform bioelectrical impedance analysis and display or otherwise export the measurements, interpretations of the measurements and/or other data via a display on the ring.

In other embodiments, the ring comprises components for communicating data via a wireless communication connection, such as Bluetooth, to an external computing device, such as a smartphone, that performs all or a portion of the measurement, interpretation and/or display functions for body fat estimation.

In any case, the present invention provides a particularly compact, error-resistant wearable device, with a unique configuration designed to provide enhanced accuracy in performing BIA. The sensing system includes a ring body sized to mate and fit snugly with, a common range of human finger sizes. As the device is configured to mate with a human finger, it is capable of measuring body fat, lean body mass, body water, etc. of many users, including those with prosthetic legs or who are wheelchair bound that cannot use many BIA devices, such as body weight scales, which require the user to assume a standing position atop the scale.

The device provides the user with a convenient measurement experience by placing the index finger and the thumb of one hand on an outer electrode positioned on an outer surface of the ring body while an electrode arranged on an interior surface of the ring body contacts skin of the wearer's finger on the other hand. The device calculates the bioelectrical impedance of the human body based on the applied current and measured voltage, measured across the human body between the left and right hands, when used as intended.

In embodiments in which the sensor readings are interpreted at the ring body, the associated data may be displayed via a display device of the ring. In embodiments in which sensor readings are interpreted at an external computing device, the associated data is then wirelessly transmitted (via associated transmission hardware housing in the ring body) to a smartphone or other external computing device, e.g., via Bluetooth, in order to estimate the body fat based on the user's personal profile (i.e., height, weight, and gender).

### BRIEF DESCRIPTION OF THE FIGURES

The following detailed description of various embodiments of the invention will be better understood when read in conjunction with the appended drawings. For the purpose of illustrating the invention, certain embodiments are shown in the drawings. It should be understood, however, that the invention is not limited to the precise arrangements and instrumentalities of the embodiments shown in the drawings.

FIG. 1A is a schematic diagram of an exemplary apparatus for analyzing body composition comprising a wearable device and an external computing device;

FIG. 1B is a schematic diagram illustrating architecture of an exemplary system;

FIG. 2 illustrates an exemplary microcontroller assembly comprising sensors for use as part of the exemplary apparatus;

FIG. 3 is a perspective view of an alternative exemplary embodiment of the finger-wearable device; and

FIG. 4 is a top view of the finger-wearable device of FIG. 4;

FIG. 5 is a side view of the finger-wearable device of FIG. 4;

FIG. 6 is a perspective view of an alternative exemplary embodiment of the finger-wearable device; and

FIG. 7 is a top view of the finger-wearable device of FIG. 4; and

FIG. 8 is a side view of the finger-wearable device of FIG. 4.

### DETAILED DESCRIPTION

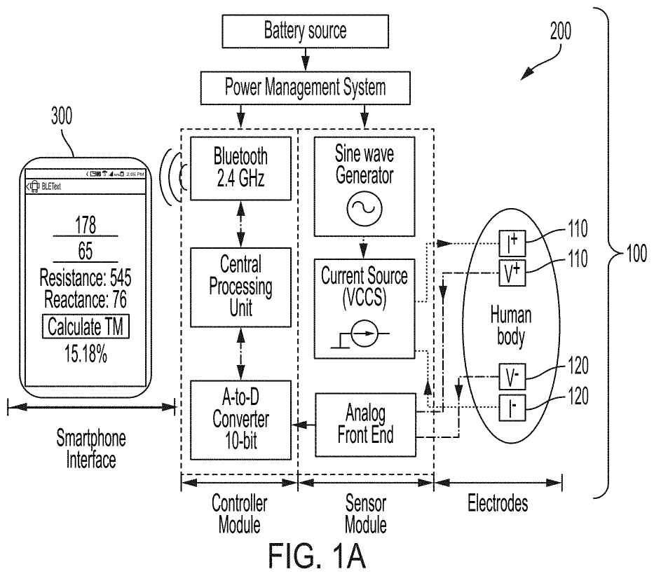

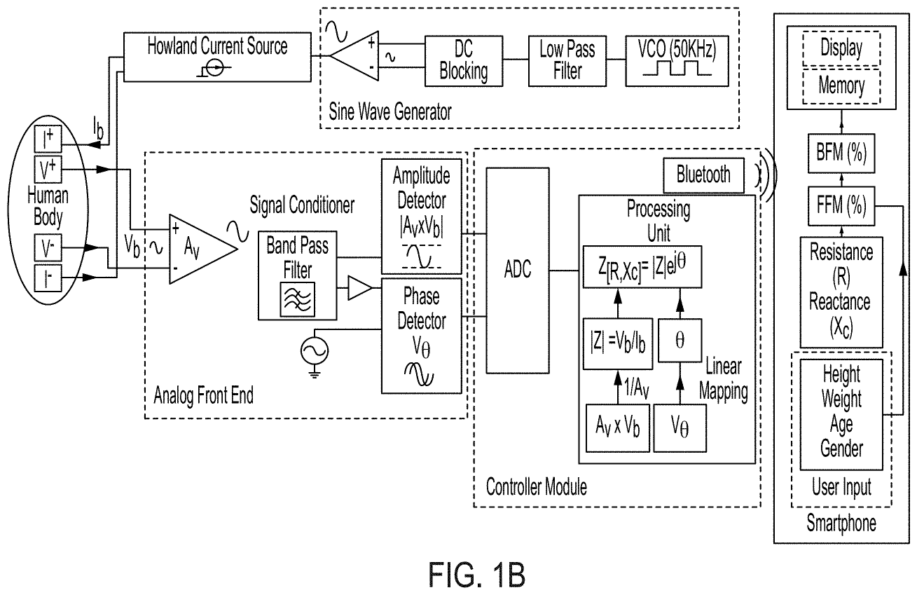

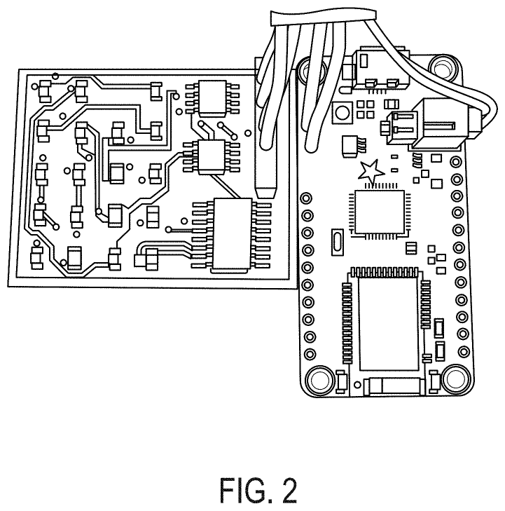

Referring now to FIG. 1A, a simplified block diagram of the ring-based analyzer is provided. This exemplary system has four primary components: electrodes, a sensor module, a controller module, and a smartphone interface. The electrodes on the ring are used to interface the sensor module with the human body. The microcontroller computes the body impedance by processing the data acquired from the sensor module. The calculated impedance is then transmitted to a connected smartphone via Bluetooth for body fat estimation.

Referring now to FIG. 1B, detailed architecture of an exemplary ring system is shown. By way of illustrative, non-limiting example only, this exemplary sensor module includes a wave generator to provide a 50 kHz sinusoidal signal, which is a suitable frequency for BIA measurement. The 50 kHz frequency may be advantageous as it is close to the characteristic frequency of biological tissues, at which the reactance reaches its maximum value, and because it is a robust frequency against various measurement artifacts. A voltage-controlled current source is designed to convert the sine wave voltage signal into a 900 μA current signal. The current source is of great significance due to safety reasons, as human safety is assured in vivo by limiting the magnitude of the applied current instead of voltage. In bioimpedance analysis, I≤1 mA is generally considered as a safe and widely accepted range for electrical current. The current source uses a Howland circuit design due to its simple principle and comparable performance with other complicated sources. The sensor module delivers the electrical current and measures the voltage across the body by using electrodes attached to a wearable ring. A detailed explanation concerning the ring and the electrodes is provided below.

As will be appreciated from FIG. 1B, the analog front end acquires the voltage signal from the human body for further analysis. The analog front end consists of an instrumentation amplifier which measures the voltage across the human body (V_{b}), amplifies it, and rejects common mode noises. The next stage of the analog front end is the signal conditioner which is used to remove interfering signals such as electrocardiogram (ECG), electromyogram (EMG), and high-frequency noises caused by electrical activities in the body. The filtered signal is then passed through a high precision amplitude detector to compute the amplitude of the signal (|Aᵥ·V_{b}|) obtained from the body. The filtered signal is simultaneously fed into a phase detector which calculates the phase difference induced by the negative reactance of the body. A phase-locked loop device, CD4046 from Texas Instruments, is used to generate the sinusoidal signal due to its built-in phase comparator and a voltage-controlled oscillator (VCO).

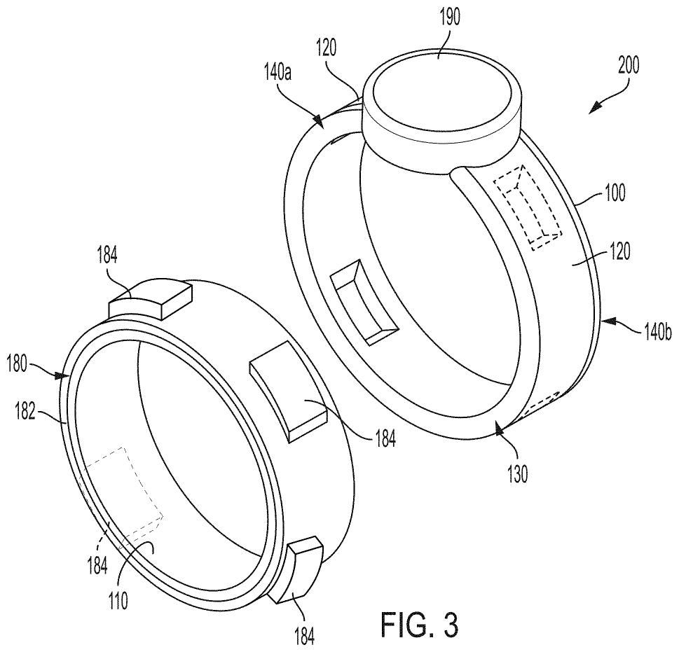

Referring again to FIG. 1B, the system further includes a controller module, which is responsible for digital signal processing and wireless communication. By way of example, an exemplary controller module may be built on Adafruit Feather 32u4 which consists of an ATmega32u4 microcontroller (8 MHz and 3.3V logic) integrated with a Bluetooth module (2.4 GHz). FIG. 3 illustrates an exemplary microcontroller interfaced with an exemplary sensor module. The exemplary microcontroller has a built-in 10-bit analog-to-digital converter to digitize the signal acquired from the analog front end. The collected signals are then processed by the microcontroller to compute the body impedance.

In this exemplary embodiment, the impedance magnitude is calculated by dividing the measured voltage (V_{b}) by the injected current (I_{b}). The phase change caused by the reactance of the body is calculated from the phase voltage (V_{θ}) by linear mapping. As known in the art, the impedance can be calculated as:

Z=|Z|e^{jθ} (1)

where Z represents the magnitude of the impedance while θ is the phase difference between the injected and measured signals. The complex impedance, in a Cartesian form, can be written as:

Z=R−jX_{c} (2)

where R represents the resistance caused by the fluids (ICF and ECF) and −X_{c} is the negative reactance imposed by cell membranes. The relationship of |Z|, R, X_{c}, and θ can be expressed as:

$$\begin{matrix}\vert Z \vert = \sqrt{{R}^{2} + {X}_{c}^{2}} & ( 3 ) \\ θ = {tan}^{- 1} ( - \frac{{X}_{c}}{R} ) & ( 4 )\end{matrix}$$

Referring again to FIG. 1A, the exemplary system further includes a smartphone application. By way of example, Android Studio may be used to develop a suitable smartphone application and interface for body fat calculations, data management, and tracking of user records. The following equation is known in the art for fat-free mass is utilized for estimating the fat mass, and may be used here:

$$\begin{matrix}{FFM}_{k g} = - 4.104 + 0.518 \times \frac{{Height}^{2}}{Resistance} + 0.2 \; 31 \times Weight + 0.130 \times Reactance + 4.229 \times sex & ( 5 ) \\ F {M}_{\%} = ( 1 - \frac{{FFM}_{k g}}{Weight} ) \times 1 \; 00 \% & ( 6 )\end{matrix}$$

where height is in cm and weight is in kg. For male sex=1 and for female sex=0. The resistance and reactance values are acquired wirelessly from the analyzer.

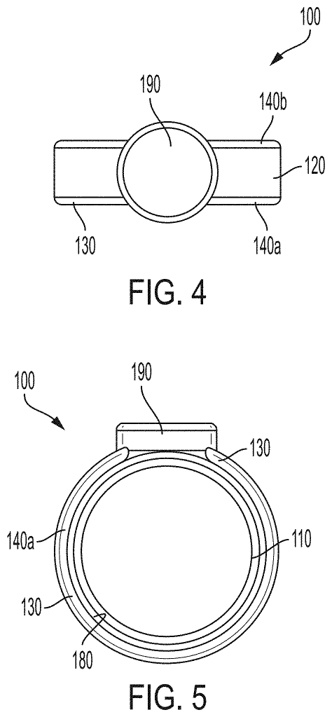

Referring now to FIGS. 3-5, an exemplary embodiment of an exemplary finger-wearable sensing system 200 is shown. The system includes a sensor and a controller module. In this exemplary embodiment, all of the sensor and the controller module are housed in the ring 100, or the electrodes and at least some computing and/or communication components are housing in the ring 100, and other computing and communication components are housed in a separate smartphone or other computing device 300, as shown in FIG. 1A.

As shown in FIGS. 1A, 3-5, the finger-wearable ring 100 includes a plurality, e.g. two, electrodes arranged in a particular configuration about a ring body 130. In this embodiment, two electrodes are used. In other embodiments, each electrode may be comprised of multiple discrete electrodes. In any case, the two electrodes are used as positive and negative terminals (respectively) to deliver high-frequency alternating current from a current source, such as a battery, housed within the ring body 130. Further, the two electrodes are used as positive and negative terminals (respectively) for sensing voltage.

he electrodes are preferably formed of a conductive metal, such as copper, which is inelastic. The electrodes are spaced from one another and separated by insulative material, such as silicone, as shown in FIG. 3. Preferably, the electrodes span at least 50%, at least 60%, at least 70%, at least 80%, or at least 90% of the interior or exterior surface of the ring.

Referring again to FIG. 3, one of the electrodes 110 is positioned on an interior surface of the ring 100, which virtually ensures robust, reliable, and error-resistant electrical connection between the electrodes and the skin of a finger of the user, without substantial interference by body hair, etc. that may interfere with discrete electrodes in a bracelet intended to be worn about the wrist, for example. Thus, this electrode is arranged to make reliable and consistent contact with the finger, and thus to promote accurate and reproducible measurements. For example, this electrode 110 may be used as the negative terminal for current (I⁻) and voltage (V⁻).

The other electrode 120 is positioned on an exterior surface of the ring 100. In one embodiment, the electrode is arranged in a position that is spatially separate from adjacent fingers, to avoid conductive contact therewith. This can be facilitated at least in part by the provision of a relatively large display screen 190 or other enlarged portion of the sensor system that naturally, or through user effort, aligns to a top of the finger when worn, and thus serves to orient the outer electrode in the desired position, as the outer electrode is arranged in a predefined spatial relationship relative to the display screen/enlarged portion. For example, this electrode 1210/22/20190 may be used as the positive terminal for current (I⁺) and voltage (V⁺).

More particularly, the ring 100 comprises a ring body 130, and a circuitry body 180 configured to be complementary in shape and nest with the ring body 130. More particularly, the circuitry body 180 includes a flexible printed circuit board 182 that provides electrical connections among electronic components 184 of the device. The electronic components 184 may be arranged on the outer surface of the circuitry body 180, and the circuitry body 180 may be shaped and dimensioned to correspond to an internal portion of the ring body 130. The circuitry body 180 may be partially or fully housed within material of the ring body 130. The ring body 130 may define internal cavities or openings 135 to receive the electronic components 184, while also encapsulating the components and/or admitting passage of wires/conductors within the ring body 130. The ring body 130 may thereby house the critical components of the sensing system inside a dust- and moisture-protected printed enclosure (e.g., 100% fill) to assist the device in functioning properly in harsh environments. The inner electrode 110 is supported on the inner surface of the circuitry body 180, as shown in FIG. 3.

In the exemplary embodiment of FIGS. 3-5, the ring body 130 includes a closed loop constructed of an electrically insulative and elastic material, such as silicone. Accordingly, portions of the closed, e.g., continuous, loop that are not fixed to the electrodes or other components are free to stretch to adapt to and conform closely with various fingers of a range of different sizes, and to urge the inelastic electrodes into contact with the user's skin to ensure robust and reliable conductive contact.

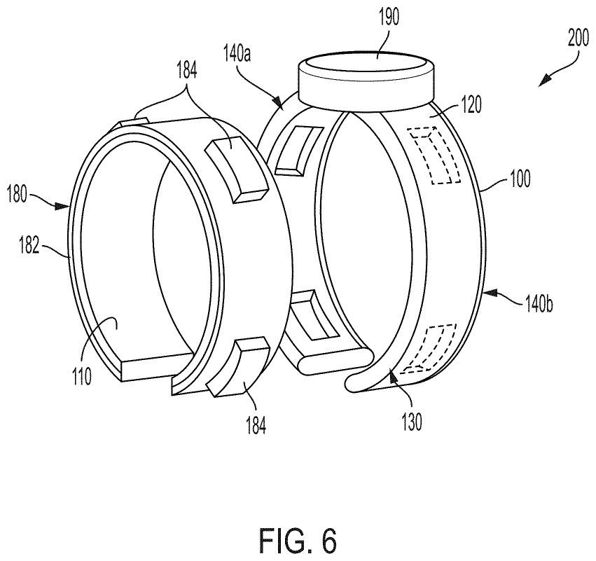

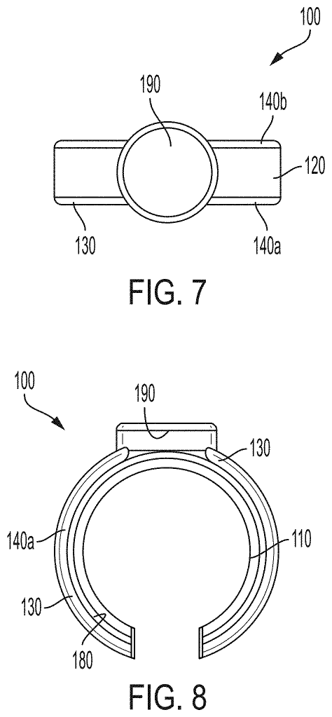

In the exemplary embodiment of FIGS. 6-8, the ring body 130 includes a substantially-closed constructed of an electrically insulative and elastic material, such as silicone. The substantially-closed loop includes sufficient structure to encircle most of, and mate securely with, a finger, but has an opening defined between ends of the ring body, so that the split-ring can easily expand or resile to adapt to and conform closely with various fingers of a range of different sizes, and to urge the inelastic electrodes into contact with the user's skin to ensure robust and reliable conductive contact.

The pairs of electrodes are electrically insulated from one another by non-conductive, insulative material of the ring body 130, e.g., via a continuous lateral shoulders 140a, 140b, as best shown in FIG. 3. The lateral shoulders extend around the periphery of the closed loop and enclose lateral edges of the electrode on the inner surface of the ring body from the electrode on the outer surface of the ring body.

Components for providing signals via the electrodes and for sensing current and voltage across the electrodes, and methods for interpreting resulting measurements to provide BIA metrics, are well-known in the art, and thus are not discussed in detail herein. Any suitable known components and methods may be used for this purpose.

In one embodiment in which each electrode 110, 120 comprises a discrete pair of electrodes (e.g., one discrete electrode for each of the positive and negative current (I) terminals, and one discrete electrode for each of the positive and negative voltage (V) terminals), the ends of the electrodes of the first pair of inelastic electrodes are spaced from one another about a periphery of the ring body to define a limited elastic deformation zone therebetween, such that a finger larger than the ring body in an unstretched state causes the ring body to stretch within the limited elastic deformation zone, but not in zones fixed to the inelastic electrodes, to accommodate the finger, and to cause the electrodes to effectively clamp the finger therebetween.

The device thereby provides the user with a convenient measurement experience by placing the index finger and the thumb on the outer electrode 110 positioned on the outer surface of the ring body, e.g., after splaying the fingers to avoid contact of the outer electrode 110 with adjacent fingers, and after initiating a measurement sequence, e.g., via the display or via software on a smartphone 300 communicating via wireless transmission with the electronic components of the ring 100 (see FIG. 1). The sensor and controller, housed at least in part in the ring body and/or in collaboration with the smartphone 300, calculates the bioelectrical impedance of the human body based on the applied current and measured voltage, measured across the human body between the left and right hands, when used as intended.

In embodiments in which the sensor readings are interpreted at the ring body, the associated data may be displayed via a display device 190 of the ring 100. In embodiments in which sensor readings are interpreted at an external computing device, the associated data is wirelessly transmitted (via associated transmission hardware housed in the ring body 130) to a smartphone or other external computing device 300, e.g., via Bluetooth, in order to estimate the body fat based on the user's personal profile (i.e., height, weight, and gender). Resulting BIA metric data may be displayed at the external computing device 300, e.g., via a suitable “app”, or may be subsequently transmitted back to the ring 100 for display via the ring's display device 190.

The disclosures of each and every patent, patent application, and publication cited herein are hereby incorporated herein by reference in their entireties.

Although the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A sensing system for analyzing body composition based on bioelectrical impedance analysis, the sensing system comprising:
a ring body comprising a loop of an electrically insulative material, the loop having an inner surface and an outer surface;
a first electrode supported on the inner surface of the ring body;
a second electrode supported on the outer surface of the ring body;
the first and second electrodes being terminals for measurement of current and voltage; and
a controller configured to measure impedance based on a voltage-current ratio by making an alternating current flow between the electrodes with a current generator, and to read a voltage difference therebetween.

2. The sensing system of claim 1, wherein the controller comprises:
an impedance measuring circuit;
a microprocessor for processing data received from the impedance measuring circuit;
an amplifier and A/D converter for interfacing the impedance measuring circuit to a microprocessor.

3. The sensing system of claim 2, wherein the controller, the amplifier and the microprocessor are supported on the ring body.

4. The sensing system of claim 2, wherein the controller, the amplifier and the microprocessor are housed within the ring body.

5. The sensing system of claim 2, wherein the controller further comprises wireless communication circuitry, and wherein the wireless communication circuitry is configured to communication via wireless communication with a wireless communication device.

6. The sensing system of claim 2, wherein the wireless communication device is a smartphone.

7. The sensing system of claim 1, wherein the ring body comprises:
an outer ring body constructed of an electrically insulative material, the first electrode being supported on the outer surface of the outer ring body; and
a flexible circuit board substrate supporting operatively interconnected electronic components of the controller, the flexible circuit board having an outer surface facing the outer ring body and an inner surface opposing its outer surface, the second electrode being supported on the inner surface of the flexible printed circuit board.

8. The sensing system of claim 1, further comprising a display fixed to the ring body, the display being operatively connected to the controller for displaying data relating to measured impedance.

9. The sensing system of claim 1, wherein the ring body is formed as a continuous closed loop.

10. The sensing system of claim 1, wherein the ring body is formed of elastic material permitting the ring to expand by stretching of the elastic material.

11. The sensing system of claim 1, wherein the ring body is formed of elastic silicone material.

12. The sensing system of claim 1, wherein the ring body is formed as a discontinuous, substantially-closed loop, having end portions separated by an open gap permitting the ring body to expand by spreading of the end portions.

13. A sensing system for analyzing body composition based on bioelectrical impedance analysis, the sensing system comprising:
a ring body comprising at least a partial loop of an electrically insulative material, the partial loop having an inner surface and an outer surface;
a first electrode supported on the inner surface of the ring body;
a second electrode supported on the outer surface of the ring body;
the first and second electrodes being terminals for measurement of current and voltage; and
a controller configured to measure impedance based on a voltage-current ratio by making an alternating current flow between the electrodes with a current generator, and to read a voltage difference therebetween.

14. The sensing system of claim 13, wherein the controller comprises a sine wave generator, a current source, and an analog front end.

15. The sensing system of claim 14, wherein the controller further comprises an analog to digital converter and a central processing unit.

16. The sensing system of claim 15, wherein the controller further comprises a wireless communication module for communicating data via wireless transmission to a wireless communication device.

17. The sensing system of claim 16, wherein the wireless communication device comprises a smartphone, and wherein the smartphone comprises application software for performing body impedance analysis calculations based on measured voltage and current sensed via the first and second electrodes.

18. A sensing system for analyzing body composition based on bioelectrical impedance analysis, the sensing system comprising:
a ring body comprising a closed loop of an electrically insulative and elastic material, the closed loop having an inner surface and an outer surface;
a first pair of inelastic conductive electrodes, each electrode of the first pair of conductive electrodes being supported on the inner surface of the ring body;
a second pair of inelastic conductive electrodes, each electrode of the second pair of conductive electrodes being supported on the outer surface of the ring body;
one of the first and second pairs of inelastic conductive electrodes being voltage electrodes and another of the first and second pairs of inelastic conductive electrodes being current electrodes;
a controller comprising an impedance measuring circuit for measuring impedance based on a voltage-current ratio by making an alternating current flow between two of the electrodes with a current generator, and for reading a voltage difference therebetween;

19. The sensing system of claim 18, wherein the controller comprises:
a microprocessor for processing data received from the impedance measuring circuit;
an amplifier and A/D converter for interfacing the impedance measuring circuit to the microprocessor; and
a display unit for displaying the results processed by the microprocessor.

20. The sensing system of claim 18, wherein the controller, the amplifier and the microprocessor are supported on the ring body.

21. The sensing system of claim 18, wherein the controller, the amplifier and the microprocessor are housed within the ring body.

22. The sensing system of claim 18, wherein the electrodes of the first pair of inelastic conductive electrodes span at least 50% of the interior surface of the ring body.

23. The sensing system of claim 18, wherein ends of the electrodes of the first pair of inelastic electrodes are spaced from one another about a periphery of the ring body to define a limited elastic deformation zone therebetween.

24. The sensing system of claim 18, wherein the electrodes of the first pair of inelastic conductive electrodes span no more than 20% of the interior surface of the ring body.

25. The sensing system of claim 18, wherein the electrodes of the first pair of inelastic conductive electrodes are positioned between 60 degrees and 120 degrees apart from one another about a periphery of the ring body.
